(19) United States
(12) Patent Application Publication
(10) Pub. No.: US 2009/0005866 A1
(43) Pub. Date: Jan. 1, 2009

(54) FIRST ELASTIC HINGE ACCOMMODATING INTRAOCULAR LENS

(75) Inventor: J. Stuart Cumming, Laguna Beach, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(73) Assignee: C&C VISION INTERNATIONAL LIMITED

(21) Appl. No.: 11/926,666

(22) Filed: Oct. 29, 2007

Related U.S. Application Data
(60) Provisional application No. 60/894,631, filed on Mar. 13, 2007.

(51) Int. Cl. A61F 2/16

(52) U.S. Cl. 623/6.39; 623/6.11; 623/6.38; 623/6.56

(57) ABSTRACT

An accommodating lens where the optic is moveable relative to the ends of the extended haptic portions. The lens comprises an optic made from a flexible material combined with haptics capable of multiple flexions without breaking. The haptics having in longitudinal cross section wide and deep hinges adjacent the optic to better allow the elastic hinges to “stretch” when the optic is subjected to posterior pressure thus allowing the optic to move forward relative to both the outer and inner ends of the haptics. When this movement is combined with the movement of the optic relative to the outer ends of the haptics and the anterior movement of the whole lens, the refractive power of the eye is further enhanced.

### CROSS-REFERENCE TO RELATED APPLICATIONS

This application claims priority from Ser. No. 60/894,631 filed Mar. 13, 2007, the disclosure of which is incorporated herein by reference.

### BACKGROUND

Intraocular lenses have for many years had a design of a single optic with loops attached to the optic to center the lens and fixate it in the empty capsular bag of the human eye. In the mid '80s plate lenses were introduced, which comprised a silicone lens, 10.5 mm in length, with a 6 mm optic. These lenses could be folded but did not fixate well in the capsular bag, but resided in pockets between the anterior and posterior capsules. The first foldable lenses were all made of silicone. In the mid 1990s an acrylic material was introduced as the optic of lenses. The acrylic lens comprised a biconvex optic with a square edge into which were inserted loops to center the lens in the eye and fixate it within the capsular bag.

Recently accommodating intraocular lenses have been introduced to the market, which generally are modified plate haptic lenses. A plate haptic lens may be referred to as an intraocular lens having two or more plate haptics joined to the optic.

Flexible acrylic material has gained significant popularity among ophthalmic surgeons; however some acrylic materials are incapable of multiple flexions without fracturing. In 2003 more than 50% of the intraocular lenses implanted had acrylic optics. Flexible hydrogel and collamer lenses have also been introduced.

The advent of an accommodating lens which functions by moving along the axis of the eye by repeated flexions somewhat limited the materials from which the lens could be made. Silicone is the ideal material, since it is flexible and can be bent probably several million times without showing any damage. Additionally a groove or hinge can be placed across the plate adjacent to the optic as part of the lens design to facilitate movement of the optic relative to the ends of the haptics.

### SUMMARY OF THE INVENTION

According to a preferred embodiment of this invention, an accommodating lens comprises a lens with a flexible solid optic attached to which are two or more extended portions. The optic may be biconvex, polyspheric, aspheric or have a Fresnell surface. The extended portions, haptics, can be plates or loops FIGS. 4, 5, & 6, which can be open or closed, each capable of multiple flexions without breaking. The haptics preferably having fixation and centration features at their distal ends. The extended portions are designed such that upon constriction of the ciliary muscle with its associated increase in vitreous cavity pressure, the extended portions are prevented from moving peripherally or outwards. This can be accompanied by making the distal end narrower than the proximal end, or by extending portions having parallel sides. The haptics are prevented from moving peripherally since the wider haptic cannot move into the smaller pocket formed by fusion of the anterior and posterior capsules. Such a lens design upon ciliary muscle contraction therefore moves centrally and posteriorly further increasing vitreous cavity pressure. Hinges or grooves across the extended portions adjacent to the optic facilitate the anterior and posterior movement of the optic relative to both ends of the extended portions by stretching of the elastic base of the hinge with ciliary muscle contraction and an increase of vitreous cavity pressure. This is additive to the anterior movement of the optic relative to the outer ends of the haptics by a steepening of the angle between the lens optic and haptics. Conversely the plate haptics may have a narrow proximal end or parallel sides.

In addition, with constriction of the ciliary muscle and relaxation of the zonules, the peripheral radial pull on the lens is reduced and the fibrosed capsular bag can then exert a central radial force longitudinally on the lens which with an increase in vitreous cavity pressure can cause a change in shape of the optic such that it is additive to the optic movement and adds power to the change in the eye's refraction. This can occur by either deformation of the optic or by an increase in the thickness of the optic center with a decrease in its radius of curvature.

The accommodating power change of the accommodating IOL upon ciliary muscle contraction can therefore be the combined results of four factors.

a) The anterior movement of the whole lens such that occurs in the human crystalline lens.

b) The movement of the optic relative to the outer ends of the haptics by a change in the angle between the optic and haptics.

c) The anterior movement of the optic relative to both the outer and inner ends of the haptics by stretching of the elastic base of the hinge.

d) Deformation of the thin lens optic.

The various mechanisms can act alone or in combination and are mainly dependent on the design of the optic and haptics. The haptics can be either a plate or loop design, and the loops either open or closed. The preferable design is a plate.

A plate design with a wide proximal end hinged adjacent to the optic cannot move peripherally upon ciliary muscle contraction and the resultant increase in vitreous cavity pressure since the pocket formed between the fused anterior and posterior capsules is narrower peripherally and is too small to allow the wider plate to move peripherally into it. With ciliary muscle contraction, the plate of this design moves centrally and since the lens within the eye is vaulted posteriorly, the plates proximal end also moves posteriorly further increasing the vitreous cavity pressure. The optic then moves anteriorly relatively to both the outer and inner plate ends by stretching of the thin hinge base.

Accordingly, features of the present invention are to provide an improved form of accommodating lens.

### BRIEF DESCRIPTION OF THE DRAWINGS

FIG. 1 is a front elevational view of a preferred embodiment of the present invention.

FIG. 2 is a side view.

FIG. 3 is a detail view of a modified hinge.

FIGS. 4-6 illustrate alternative embodiments with or without hinges.

FIG. 7 is a variation of the lens.

According to the present invention, the optic is of a foldable, flexible silicone, acrylic, collamer or hydrogel material and the haptic plates are of a foldable material that will withstand multiple foldings without damage, e.g., silicone, hydrogel, collamer. Preferably, the end of the plate haptics essentially have T-shaped fixation devices and may be hinged to the optic.

### DESCRIPTION OF THE PREFERRED EMBODIMENT

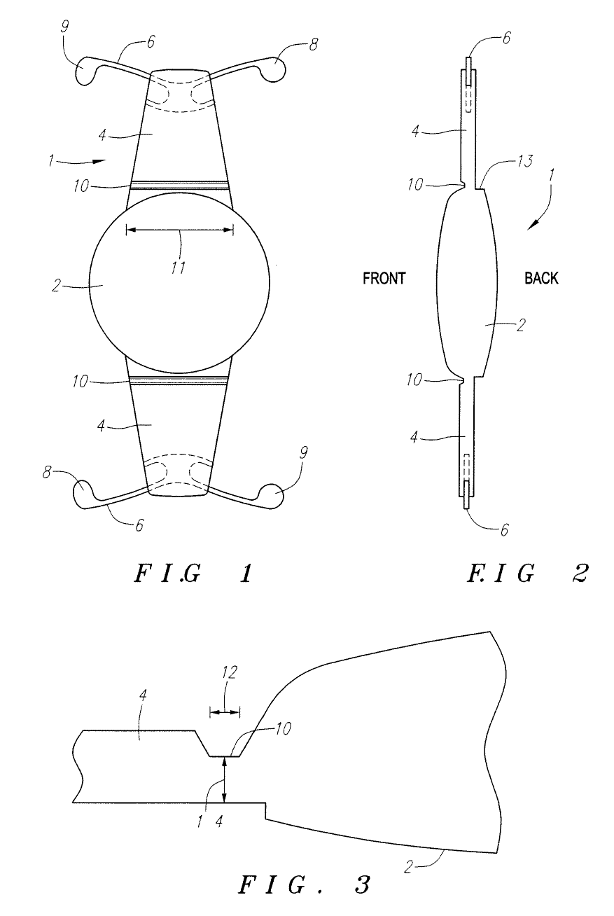

Turning now to the Figures, a preferred embodiment is illustrated in detail in FIGS. 1-3 comprising an intraocular lens 1 formed as a flexible solid optic 2 with a 360° posterior square edge preferably made of silicone, and flexible extending portions 4 of any suitable form but preferably triangular plate haptics with the wide base adjacent to the optic which are capable of multiple flexations without damage and formed, for example, of silicone. The optic 2 and haptics 4 preferably are uniplanar until implanted into the eye, and two haptics 4 extend distally from opposite sides of the optic 2. Fixation and centration fingers 6 are provided at the distal ends of the hinged haptics 4.

A typical length for the lens is 10.0-12.0 mm, and the optic 2 typically is a 4.5-6.0 mm diameter optic. The fingers 6 preferably are approximately 5.0 mm wide and comprise four-point fixation loops. The ends 8 have a slightly different configuration and aid in indicating to the surgeon that the lens is right side up with the hinges in a proper position.

Importantly, the haptics 4 have a triangular shape, wider adjacent to the optic, and narrower at the outer ends. Hinges 10 are provided between the haptics 4 and the outer periphery of the optic 2, and it is particularly desirable to have a wide elastic base 12 to the hinge to allow the optic 2 to move forward more by stretching of the thin and wide hinge base in the longitudinal axis of the lens with the increase in vitreous cavity pressure. The wide hinge base in the longitudinal axis of the lens allows more anterior movement than in current designs such as for example that shown in U.S. Pat. No. 6,398,126. A typical hinge width 11 at the junction of the haptic with the optic is 3.0-5.0 mm, and preferably with a hinge base width longitudinally as indicated by arrow 12 of 0.06-0.4 mm, and preferably 0.12 mm, as seen in FIG. 3. The wider hinge base 12 stretches like an elastic band to facilitate greater anterior movement of the optic 2.

The hinges 10 are on the anterior side and the round end 8 of loops 6 on the right as seen in FIG. 1 indicates that the hinge is uppermost. End 8 is round.

Preferably the optic and plate haptics are silicone and the loops 6 are polyimide.

There can be a sharp edge around the posterior surface of the optic 2, to reduce the migration of cells across the posterior capsule of the lens postoperatively and thereby reduce the incidence of posterior capsular opacification and the necessity of YAG posterior capsulotomy.

As is well known in the art, the intraocular lens 1 such as that in the drawings is implanted in the capsular bag of the eye after removal of the natural lens. The lens is inserted into the capsular bag through a generally circular opening torn in the anterior capsular bag of the human lens after passing through a small opening in the cornea or sclera. The outer ends of the haptics 4, or loops 6, are positioned in the cul-de-sac of the capsular bag. The outer ends of the haptics, or the loops, are in close proximity with the bag cul-de-sac, and loops are deflected centrally to conform with the inner surface of the capsular bag. The ends or knobs of the loops are provided on the outer end portions of the loops 6 for fixation to secure the lens in the capsular bag or cul-de-sac with fibrosis, which develops in the capsular bag following the surgical removal of the central lens cortex and nucleus.

The inner ends of the loops 6 may be either integrally formed from the same material as the haptics 4 or the loops may be of a separate material such as polyimide. The loops, if formed of a separate material, are molded into the terminal portions of the haptics 4.

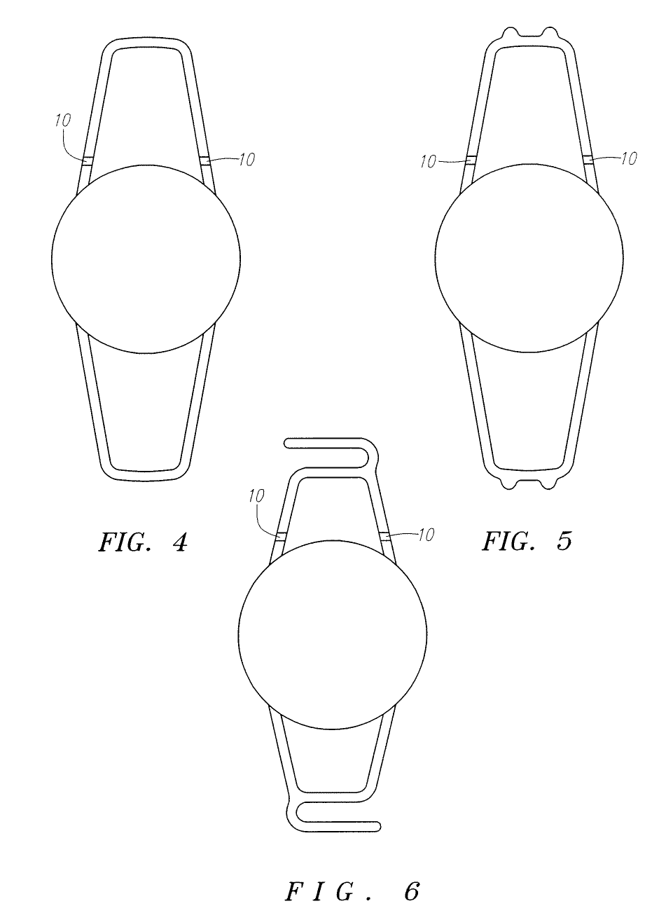

FIGS. 4-6 show alternative forms of haptics. The haptics can be with or without hinges on either or both haptics. Hinges are illustrated on the upper haptics in FIGS. 4, 5 and 6.

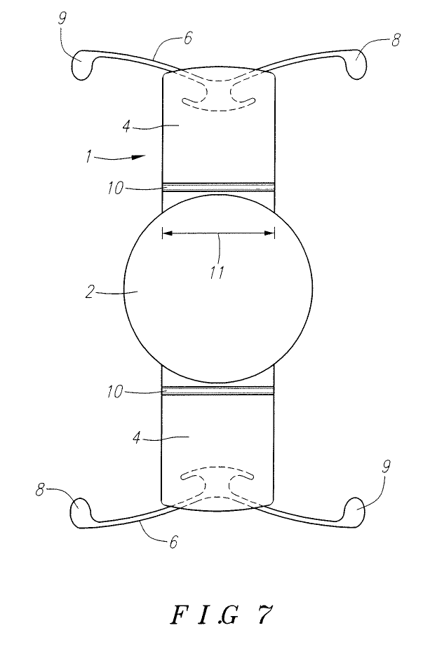

FIG. 7 is a variation of the lens of FIG. 1 and wherein the haptics 4 have parallel sides.

Accordingly, there has been shown and described a lens that ideally comprises a silicone optic and silicone haptic plates with loops at their distal ends that can be of a different material than the plate, and provide fixation and centration of the lens in the eye. The haptics designed for movement centrally and posteriorly along the tunnel formed by the fusion of the anterior and posterior capsules of the human capsular bag. The lens having wide elastic bases to the hinges that stretch in the longitudinal axis of the lens like a rubber band to allow the optic to move by flexion of the hinge and stretching of its wide elastic base.

Various changes, modifications, variations, and other uses and applications of the subject invention will become apparent to those skilled in the art after considering this specification together with the accompanying drawings and claims. All such changes, modifications, variations, and other uses of the applications which do not depart from the spirit and scope of the invention are intended to be covered by the claims which follow.

What is claimed is:

1. An accommodating intraocular lens comprising a flexible solid optic and attached flexible extended portions comprising haptics, designed such that the optic can move backward and forward in pockets formed by the fusion of the anterior and posterior capsules of the eye relative to the extended portions and whereby the haptics adjacent to the optic have a thinned elastic area such that upon an increase in posterior pressure, the thinned elastic area can stretch to further aid anterior movement of the optic relative to both the outer and inner ends of the haptics.

2. A lens according to claim 1 wherein the haptics are relatively wide adjacent the optic and are narrower distally.

3. A lens according to claim 1 wherein the haptics have parallel sides.

4. A lens according to claim 1 wherein the haptics are relatively thin adjacent to the optic.

5. A lens according to claim 1 wherein the haptics have a thinned area adjacent to the optic.

6. A lens according to claim 5 wherein the thinned area is a hinge and is V-shaped.

7. A lens according to claim 5 wherein the thinned area is a hinge and is V-shaped and has a wide base connecting the two sides of the hinge.

8. A lens according to claim 5 wherein the thinned area is a shallow groove.

9. A lens according to claim 1 wherein one or more fixation/centration fingers are on the ends of the extended portions.

10. A lens according to claim 9 wherein the fixation/centration fingers indicate the correct side up of the lens to be inserted in the eye.

11. A lens according to claim 9 wherein the fingers are designed to extend beyond the diameter of the capsular bag and are flexible to bend to conform to the bag diameter.

12. A lens according to claim 1 where the extended portions include loops and/or fixation devices of polyimide.

13. A lens according to claim 5 where the loops have a fixation element of a different shape on their proximal ends to enhance centration and fixation of the lens within the capsular bag.

14. A lens according to claim 1 wherein the lens is made of an optical material(s) that is inert, e.g. silicone, HEMA, acrylic, or other material.

15. A lens according to claim 14 where the loops or fingers are made of a different material than the lens, e.g. polyimide, PMMA, Prolene, or the like.

16. A lens according to claim 14 where the lens optic is made of a different material than the haptics.

17. A lens according to claim 1 wherein the optic has a 360 degree square edge on its posterior surface.

18. A lens according to claim 1 wherein the flexible optic is capable of a shape change that increases its refractive power upon ciliary muscle contraction.

19. A lens according to claim 1 wherein the optic has one or both surfaces that are polyspheric.

20. A lens according to claim 1 wherein the optic has one or more surfaces that are aspheric.

21. A lens according to claim 1 where the optic size is from 3.5 to 8 mm.

22. A lens according to claim 1 where the extended portions are of the same material as the optic and loops.

23. A lens according to claim 22 where there are projections from the loops to help center or fixate the lens in the capsular bag.

24. A Lens according to claim 22 where the loops are closed at their distal ends.

25. A lens according to claim 22 where the loops are of a different material than the optic.

26. A lens according to claim 23 where the loops are open at their distal ends.
